(19) United States
(12) Patent Application Publication
(10) Pub. No.: US 2018/0117088 A1
(43) Pub. Date: May 3, 2018

(54) COMPOSITION FOR TREATING ARTICULAR CARTILAGE DEFECTS

(71) Applicants: CELLULAR BIOMEDICINE GROUP (SHANGHAI) LTD., Shanghai (CN); CELLULAR BIOMEDICINE GROUP (WUXI) LTD., Wuxi, Jiangsu (CN)

(72) Inventors: Wei CAO, Shanghai (CN); Wen WANG, Shanghai (CN); Meng LI, Shanghai (CN); Chengxiang DAI, Shanghai (CN); Li ZHANG, Shanghai (CN); Luyi ZHANG, Shanghai (CN); Fei WANG, Shanghai (CN); Suke LI, Shanghai (CN); Jia LIU, Shanghai (CN); Songbai CAI, Shanghai (CN)

(21) Appl. No.: 15/557,312

(22) PCT Filed: Mar. 10, 2016
(86) PCT No.: PCT/CN2016/076082

§ 371 Date: Jan. 16, 2018

(30) Foreign Application Priority Data
Mar. 10, 2015 (CN) 201510104870.8

(51) Int. Cl. A61K 35/28; A61K 38/38; A61K 31/728; A61P 19/02

(52) U.S. Cl. CPC A61K 35/28; A61P 19/02; A61K 31/728; A61K 38/385

(57) ABSTRACT

A composition for treating articular cartilage defects, and a manufacturing method, and a kit thereof, the composition comprising: a therapeutically effective amount of fat-derived mesenchymal progenitor cells, serum albumin solution, and sodium hyaluronate, Above 95% of the fat-derived mesenchymal progenitor cells have the surface antigen CD90, CD73, CD29, and CD49d, and express cytokines selected from TGF-β1, HGF, and VEGF. The composition has the effect of treating articular cartilage defects.

### FIELD OF THE INVENTION

The present invention relates to the field of cartilage treatment. Specifically, the present invention provides a composition for treating articular cartilage defects.

### BACKGROUND OF THE INVENTION

The knee joint hyaline cartilage is between the tibia and the femur and provides buffering and lubrication effect to the knee joint together with synovial fluid. The knee joint has poor ability to repair hyaline cartilage, and it can not spontaneously heal or regenerate when it is damaged by external force. Hyaline cartilage doesn't contain blood, nerve, and lymphatic system and therefore unable to stimulate the body's repair reaction.

The early symptoms of knee joint cartilage injury is not obvious, which are generally found by arthroscopic operation, so that it is easy to progress to more severe chronic articular cartilage injury, causing pain, swelling, etc. Serious joint cartilage damage, if have not been properly treated, can easily lead to osteoarthritis of the knee, and eventually lead to disability.

At present, the main treatment method for treating articular cartilage injury is surgery. For example, microfracture surgery that drills hole in the injured knee region to stimulate self bone marrow to derive into mesenchymal stem cells to take part in the repair of articular cartilage. However, the cartilage tissues generated after microfracture surgery are fibrocartilage, rather than hyaline cartilage, so microfracture surgery has certain effect in the short term, but the long-term follow-up has showed that the function of knee undergoing microfracture surgery has still declined.

At the same time, the self cartilage particles filling repair technology can replace articular cartilage defect site in patients, the operation is relatively simple, but the source of cartilage particles is limited, at the same time, the research showed that this treatment is not effective in the treatment of >5 cm² articular cartilage injuries.

Self cartilage cells transplant surgery is another commonly treatment method used to repair the articular cartilage of the knee, this method extracts the patient's own cartilage tissues, cultured in vitro and then transplanted into the cartilage damage sites of the knee joint again. This method has a high success rate in treating knee joint cartilage injuries, but requires improved technical conditions, while collecting the patient's own cartilage tissue is of a certain extent of risk at the same time, the cartilage cells cultured in vitro are easy to aging and differentiate thus losing the ability to repair articular cartilage injury.

Progenitor cells are a group of cells with self-renewal and differentiation potential, fat-derived mesenchymal progenitor cells are a type of adult mesenchymal progenitor cells, which is easy to obtain and less harmful to the donor, while at the same time keeping the chondrogenesis, osteogenesis and adipogenic differentiation ability, and can be amplified abundantly and maintained their differentiation ability in vitro culture environment. Therefore, it is an ideal cell type for treating knee joint cartilage injuries. However, existing progenitor cell compositions used in clinical treatment of articular cartilage defects by intra-articular injections often appear complications such as swelling and soreness of the joints. Therefore, a new type of cartilage damage repair agent is needed in this field.

### SUMMARY OF THE INVENTION

The object of the present invention is to provide an articular cartilage defect composition which is ease to use, component optimized, and can rapidly promote the repair of cartilage injury site.

In the first aspect of the invention, a composition for treating articular cartilage defects is provided, which comprises: a therapeutically effective amount of fat-derived mesenchymal progenitor cells, human serum albumin solution, and sodium hyaluronate.

In another preferred embodiment, said human serum albumin solution is 0.5-2 (v/v) % human serum albumin; preferably 0.8-1.5 (v/v) %.

In another preferred example, said human serum albumin solution is 0.5-2 (v/v) % human serum albumin Multiple Electrolytes Injection solution.

In another preferred embodiment, said sodium hyaluronate is solution, preferably 15-25 wt % sodium hyaluronate solution.

In another preferred embodiment, said treating articular cartilage defects refers to the repair of cartilage defects, preferably, said the repair of cartilage defects includes one or more features selected from the group consisting of the increase of the cartilage thickness, cartilage matrix regeneration, cartilage type II collagen hyperplasia, the inhibition of the secretion of cartilage degradation enzyme MMP-13.

In another preferred embodiment, said treating articular cartilage defects includes one or more indexes of the treatment object selected from the following group is improved: the pain score NRS-11, joint dysfunction WOMAC score, the volume of articular cartilage, the thickness of articular cartilage, bone marrow edema.

In another preferred case, said mesenchymal progenitor cells have one or more features selected from the following group:

(i) more than 95% of cells having surface antigen CD90;

(ii) more than 95% of cells having surface antigen CD73;

(iii) more than 95% of cells having surface antigen CD29;

(iv) more than 95% of cells having surface antigen CD49d.

In another preferred embodiment, more than 98% of the cells have surface antigen CD90, preferably, more than 99% of the cells have surface antigen CD90.

In another preferred embodiment, more than 98% of the cells have surface antigen CD73, preferably, more than 99% of the cells have surface antigen CD73.

In another preferred embodiment, more than 98% of the cells have surface antigen CD29, preferably, more than 99% of the cells have surface antigen CD29.

In another preferred embodiment, more than 98% of the cells have surface antigen CD49d, preferably, more than 99% of the cells have surface antigen CD49d.

In another preferred embodiment, more than 99.6% of cells have surface antigen CD90.

In another preferred embodiment, more than 99.7% of cells have surface antigen CD73.

In another preferred embodiment, more than 99.5% of cells have surface antigen CD29.

In another preferred example, more than 99.8% of cells have surface antigen CD49d.

In another preferred embodiment, said mesenchymal progenitor cells have one or more features selected from the following group:

(v) less than 2% of cells having surface antigen CD34;

(vi)) less than 1% of cells having surface antigen CD45;

(vii) less than 0.3% of cells having surface antigen Actin;

(viii) less than 0.5% of cells having surface antigen CD14;

(ix) less than 0.1% of cells having surface antigen HLA-DR.

In another preferred embodiment, less than 1% of the cells have surface antigen CD34, preferably, less than 0.5% of the cells have surface antigen CD34.

In another preferred embodiment, less than 0.5% of the cells have surface antigen CD45, preferably, less than 0.1% of the cells have surface antigen CD45.

In another preferred embodiment, said cells do not have surface antigen CD34.

In another preferred embodiment, said cells do not have surface antigen CD45.

In another preferred embodiment, said cells do not have surface antigen CD14.

In another preferred embodiment, said cells do not have surface antigen HLA-DR.

In another preferred embodiment, the concentration of fat-derived mesenchymal progenitor cells in the composition is 10⁵-10⁹/mL.

In another preferred embodiment, the concentration of fat-derived mesenchymal progenitor cells in the composition is 10⁶-10⁸/mL.

In another preferred embodiment, the concentration of fat-derived mesenchymal progenitor cells in the composition is 5×10⁵-5×10⁶/mL.

In another preferred embodiment, the concentration of fat-derived mesenchymal progenitor cells in the composition is 5×10⁶-5×10⁷/mL.

In another preferred embodiment, said fat-derived mesenchymal progenitor cells also express cytokines, and said cytokines are selected from the group consisting of TGF-β1, HGF, VEGF, or the combinations thereof.

In another preferred embodiment, the amount of cytokine TGF-β1 expressed by said fat-derived mesenchymal progenitor cells is 1000-1300 pg/ml/10⁶ cell.

In another preferred embodiment, the amount of cytokine HGF expressed by said fat-derived mesenchymal progenitor cells is 9000-10000 pg/ml/10⁶ cells.

In another preferred embodiment, the amount of cytokine VEGF expressed by said fat-derived mesenchymal progenitor cells is 300-800 pg/ml/10⁶ cells.

In another preferred embodiment, the mesenchymal progenitor cells have the chondrogenesis, osteogenesis and adipogenic differentiation ability.

In another preferred embodiment, the volume ratio of sodium hyaluronate to human serum albumin in said composition is 1-5:0.8-1.2, preferably 2-4:1, more preferably 2.5-3.5:1.

In another preferred embodiment, said composition is a unit dosage form, and the volume of said unit dosage form is ≤4 mL, preferably ≤3 mL.

In another preferred embodiment, the volume of said unit dosage form is 0.15-3 mL.

In another preferred embodiment, one unit composition is applied for per cm2 area of articular cartilage damage.

In another preferred embodiment, 5×10⁵-5×10⁶ cells are applied for per cm2 area of articular cartilage damage.

In another preferred embodiment, said composition is also used to increase the cartilage thickness,

In another preferred embodiment, said composition is also used for cartilage matrix regeneration.

In another preferred embodiment, said composition is also used for promoting cartilage type II collagen.

In another preferred embodiment, said composition is also used for the inhibition of the secretion of cartilage degradation enzyme MMP-13.

In another preferred embodiment, the fat-derived mesenchymal progenitor cells survived for 10 weeks in vivo after injection of said composition.

In the second aspect of the invention, a method for preparing the composition described in the first aspect of the invention is provided, wherein comprising the following step: mixing fat-derived mesenchymal progenitor cells, human serum albumin solution and sodium hyaluronate to prepare composition.

In another preferred embodiment, the method also comprises culturing the fat-derived mesenchymal progenitor cells, which comprises the following steps:

a) providing 30-50 ml fatty tissue;

b) digesting the fatty tissue with collagenase I;

c) discarding the digested fat and collecting the underlying deposits;

d) adding cell culture medium, mixing, removing undigested tissue blocks, and counting the cells;

f) adjusting the inoculum density and inoculating cells in T75 culture flask at 5×10⁵-2×10⁶/T75 (each T75 culture bottle inoculated with 12 ml of liposucted fat), then cultured until the adherent cells grow into colony under 35-40° C. and 1-10% CO₂.

f) digesting for 1.5-2.5 min with Trypsin EDTA solution, centrifugatng to remove digestive juice, and passaging to obtain the fat-derived mesenchymal progenitor cells.

In another preferred embodiment, the medium used for culturing fat-derived mesenchymal progenitor cells is serum-free medium or serum containing medium.

In the third aspect of the present invention a formulation for treating articular cartilage defects is provided, the formulation comprises composition described in the first aspect of the present invention as an active ingredient.

In another preferred embodiment, said formulation is injection, preferably intra-articular injection.

In another preferred embodiment, said formulation is the unit dosage forms, and the volume of said unit dosage forms is ≤4 mL, preferably ≤3 mL.

In another preferred embodiment, the volume of the unit dosage form is 0.15-3 mL.

In another preferred embodiment, one unit composition is applied for per cm² articular cartilage damage area.

In another preferred embodiment, said formulation is also used to increase cartilage thickness.

In another preferred embodiment, said formulation is also used for cartilage matrix regeneration.

In another preferred embodiment, said formulation is also used for promoting cartilage type II collagen.

In another preferred embodiment, said formulation is also used for the inhibition of the secretion of cartilage degradation enzyme MMP-13.

In another preferred embodiment, fat-derived mesenchymal progenitor cells survived for 10 weeks in vivo after injection of said formulation.

In the fourth aspect of the present invention, a reagent combination or kit is provided, which comprising:

a. fat-derived mesenchymal progenitor cells;

b. 15-25% sodium hyaluronate solution;

c. 0.5-2% human serum albumin solution;

and d. specification, the use scheme described in said specification;

and the use scheme comprises the following step: mixing components a, b, and c for treating patients with articular cartilage defects.

In the fifth aspect of the invention a method for preventing and/or treating osteoarthritis is provided, said method comprises the following step: administering composition described in the first aspect of the invention to a subject in need, or administering formulation described in the third aspect of the invention to a subject in need.

In another preferred embodiment, said method comprises the following step: injecting the joint cavity of said subject with the composition described in the first aspect of the present invention or the formulation in the third aspect of the present invention.

In another preferred embodiment, the dosage of the composition or formulation is 0.5-2×10⁶ cells for per cm² articular cartilage damage area.

In another preferred embodiment, the dosage of the composition or formulation is 1-5 mL composition or formulation inject to per cm² articular cartilage damage area.

It should be understood that, in the present invention, each of the technical features specifically described above and below (such as those in the Examples) can be combined with each other, thereby constituting new or preferred technical solutions which need not be specified again herein.

### DESCRIPTION OF FIGURES

FIG. 1 shows the result of flow cytometry test in embodiment 2;

FIG. 2 shows the chondrogenic, osteogenic and adipogenic abilities of fat-derived mesenchymal progenitor cells by the Alcian blue staining detection;

FIG. 3 shows the results of cell viability of the invention group and control group in embodiment 3;

FIG. 4 is the therapeutic effect diagram of the composition. FIG. 4A is the fast green and the counterstain safranin staining figure, FIG. 4B is the II type collagen experimental figure, and the FIG. 4C shows MMP-13 immunohistochemical experimental figure;

FIG. 5 is the experiment result of tracing fat-derived mesenchymal progenitor cells in rats; wherein, the FIG. 5A is the results of human synovium derived mesenchymal progenitor cells in the knee joint injection experiment described in the Hirie et al, Stem Cells, 2009, FIG. 5B is the results of the example 7 in the present invention.

FIG. 6 is the experimental results of therapy effect of fat-derived mesenchymal progenitor cells, the FIG. 6A shows NRS-11 score after treated with fat-mesenchymal progenitor cells composition, FIG. 6B shows the WOMAC score of articular dysfunction, and FIG. 6C shows the MRI results of articular cartilage volume.

FIG. 7 shows the results of MRI detection of case 1 in example 9: the patients are treated with knee arthroplasty surgery+the formulation of Example 5, after 7 months, the knee osteoarthritis is significantly relieved and the patella and femoral cartilage lesions disappeared.

FIG. 8 shows the results of arthroscopic observation of case 1 in Example 9: the patients are treated with knee arthroplasty surgery+the formulation of Example 5, after 7 months, there is a significant cartilage-like tissue hyperplasia in the cartilage defect sites by arthroscopy observation.

FIG. 9 shows the results of MRI detection of case 2 in example 9: the patients are treated with knee arthroplasty surgery+the formulation of Example 5, after 7 months, and the knee osteoarthritis was significantly improved and subchondral bone lesions are significantly relived.

FIG. 10 shows the results of arthroscopic observation of case 2 in Example 9: the patients are treated with knee arthroplasty surgery+the formulation of Example 5 for 7 months, and there is a significant cartilage-like tissue hyperplasia in the cartilage defect sites by arthroscopy observation.

FIG. 11 shows the results of MRI detection of case 3 in Example 3: the patients are treated with knee arthroplasty surgery+the ARTZ agent for 24 weeks, and there is no obvious improvement in the knee osteoarthritis, and subchondral bone lesions haven't been relived obviously.

FIG. 12 shows the results of arthroscopic observation of case 3 in Example 9: the patients are treated with knee arthroplasty surgery+the formulation of Example 5 for 24 weeks, and there is no significant cartilage-like tissue hyperplasia in the cartilage defect sites by arthroscopy observation.

### DETAILED EMBODIMENTS

Upon extensive and intensive studies, the inventor has unexpectedly discovered that the injection made by combining a particular type of fat-derived mesenchymal progenitor cells with hyaluronic acid can treat cartilage injury at low injection dosage (about 3 ml). The present invention is completed on this basis.

### Terms

As used herein, terms “more than” and “less than” includes the number itself, e.g., “more than 95%” means ≥95%, “less than 0.2%” means ≤0.2%.

Fat

Self fat is a good source for plastic and anti-aging treatments. fat tissue materials can be derived from the parts of waist, hips, abdomen, thighs, upper arms, etc. Those skilled in the art may obtain fat tissues by common techniques and methods including (but not limited to) suction or surgical separation, etc.

In the present invention, fat tissues or fat sources are not specifically limited. They may be derived from any part of animal or human adipose tissues, preferably human adipose tissues. Preferably, the fat tissues may be tissues from the parts of waist, hips, abdomen, thighs, upper arms, etc.

Fat-Derived Mesenchymal Progenitor Cells

As used herein, term “fat-derived mesenchymal progenitor cells”, “haMPCs” or “adipose tissue-derived mesenchymal progenitor cells” has the same meaning and can be used interchangeably.

Preferably, the fat-derived mesenchymal progenitor cells in the present invention employ is the human fat-derived mesenchymal progenitor cells; more preferably human self fat-derived mesenchymal progenitor cells.

One skilled in the art can detect the purity and differentiation degree of SVF by common methods, a preferred embodiment comprises the following steps:

a) washing fat tissue (removing blood cells): adding saline into the fat tissue to fully wash the fat tissue and separate different phase, absorbing the lower aqueous phase; the above operation is repeated until the lower liquid is clear.

b) digesting collagenase: digesting the fat tissue by adding collagenase I.

c) collecting precipitation: discarding the digested fat in upper layer, collecting the underlying precipitation to a new centrifuge tube, and adding DMEM for centrifuge and wash.

d) filtering and counting: adding DMEM, mixing, filtering to remove undigested tissue blocks, adding DMEM, and absorbing 1 ml to count cell amount and vitality.

e) inoculate and cultivate: washing by centrifugation for one time, the inoculum density was adjusted according to the amount of cells counted and inoculated into T75 culture bottle for culture.

f): passage: appearing adherence 1-2 days after inoculation, and a few adherent mesenchymal stem cells start to appear after 3 days, digesting and passaging with Trypsin EDTA solution after culturing the adherent cells to form colonies, adding 2 ml to each T75 culture bottle, digesting for 1.5-2.5 min, and then collecting the cells for cell counting, passaging at a ratio from 1:1-2 according to the primary adherent cells, after passage, the growth rate of cells increase and can be passaged again in three days. It is passaged at a ratio of 1:2-3 according to the cell growth, and P3-P7 passage cells are collected for treatment or preparation of pharmaceutical formulations.

Antigen Detection of Fat-Derived Mesenchymal Progenitor Cells

Fat-derived mesenchymal progenitor cells used in the present invention are highly purified and viability.

One skilled in the art can detect fat-derived mesenchymal progenitor cells surface antigens by common methods, such as Flow cytometry, etc.

Fat-derived mesenchymal progenitor cells have a variety of specific antigens and receptors, mainly including CD29, CD73, CD90, CD49d, etc.

The percentage of mesenchymal progenitor cells with CD90 antigen in the total mesenchymal progenitor cells is ≥92%, preferably ≥95%, more preferably ≥98%, most preferably, more than 99% of cells possess the surface antigen CD90.

The percentage of mesenchymal progenitor cells with CD73 antigen in the total mesenchymal progenitor cells is ≥92%, preferably ≥95%, more preferably ≥98%, most preferably, more than 99% of cells possess the surface antigen CD73.

The percentage of mesenchymal progenitor cells with CD29 antigen in the total mesenchymal progenitor cells is ≥92%, preferably ≥95%, more preferably ≥98%, most preferably, more than 99% of cells possess the surface antigen CD29.

The percentage of mesenchymal progenitor cells with CD49d antigen in the total mesenchymal progenitor cells is ≥92%, preferably ≥95%, more preferably ≥98%, most preferably, more than 99% of cells possess the surface antigen CD49d.

In a preferred embodiment, said cells have one or more features selected from the group: more than 99.6% of the cells having surface antigen CD90; more than 99.7% of the cells having surface antigen CD73; more than 99.5% of the cells having surface antigen CD29; and/or more than 99.8% of the cells having surface antigen CD49d.

Negative markers of fat-derived mesenchymal progenitor cells include CD34, CD45, ect. In the present invention, the percentage of mesenchymal progenitor cells with CD34 antigen in the total mesenchymal progenitor cells is ≤2%, preferably ≤1%, more preferably ≤0.5%, most preferably without CD34.

The percentage of mesenchymal progenitor cells with CD45 antigen in the total mesenchymal progenitor cells is ≤1%, preferably ≤0.5%, and more preferably ≤0.1%, most preferably without CD45.

Those skilled in the art can done routine operations such as use, treat, administrate the haMPCs by conventional methods. For example, each batch of haMPCs should pass the sterile, endotoxin and mycoplasma tests and the DNA establishing identification before it is released or used. Each batch of issued cells should meet the following requirements: cell viability ≥95%, cell purity (positive markers ≥95%, negative markers <2%), and negative in haMPCs acute toxicity and allergy test results. Each of the above should have a corresponding test report.

Cartilage Defects and Repair

Articular cartilage is hyaline cartilage, mainly composed of chondrocytes, cartilage matrix and type II collagen, it has good elasticity, friction coefficient and other mechanical properties, which is extremely important to the joint function. Joint trauma inflammation and other diseases often cause articular cartilage damage, Due to limited ability of articular cartilage regeneration, once articular cartilage is injured, it is difficult to repair, thus resulting in joint dysfunction. For a long time, how to repair the damaged articular cartilage has been one of the important subjects of orthopedic research.

In the invention, the repair of cartilage defects includes one or more indexes improvement selected from the group consisting of the promotion of the cartilage thickness, cartilage matrix regeneration, cartilage type II collagen, the inhibition of the secretion of cartilage degradation enzyme MMP-13, the reduction of symptoms such as joint effusion, spur hyperplasia, bone marrow edema, the increase of chondroplasia gene expression (preferably said chondroplasia gene is selected from the group consisting of Collegen II, TGF-beta, BMP-2, or the combinations thereof), the decrease of cartilage lytic enzyme inhibitory gene expression (preferably said cartilage degradation enzyme is selected from the group consisting of TIMP1, TIMP2, or the combinations thereof), and the increase of generating cartilage cells signaling pathway gene expression (preferably said generating cartilage cells Signaling pathway gene is selected from the group consisting of p-ERK1/2, Ihh, or the combination thereof).

In particular, in clinical treatment, said repair includes improvement of the treated subject in one or more indexes in selected from the group consisting of the pain score NRS-11, joint dysfunction WOMAC score, the volume of articular cartilage, the thickness of articular cartilage, bone marrow edema.

Sodium Hyaluronate

Sodium hyaluronate is a physiologically active substance found widely in animals and humans. It is found in human skin, synovial fluid, umbilical cord, aqueous humor, and vitreous humor. With a molecular weight of 500000˜730000 Dalton, the sodium hyaluronate solution has high viscoelasticity and profiling, and is often used as an adjunct drugs for ophthalmic surgery. It can also be injected into the abdominal cavity after abdominal surgery to reduce the postoperative intestinal adhesion. It can also be injected into the joint cavity to reduce joint surface friction and relieve joint pain. Bladder perfusion can also be used as a temporary substitute for the lack of glucosamine protected layer in the bladder epithelium.

Pharmaceutical Compositions and the Use Thereof

The present invention also provides an injectable composition, which comprises effective amounts of mesenchymal progenitor cells, and pharmaceutically acceptable carriers.

Usually, mesenchymal vessel-layer cells and mesenchymal progenitor cells can be prepared in nontoxic, inert and pharmaceutically acceptable aqueous carrier medium, such as saline, of which the pH is usually about 5-8, preferably, about 7-8.

As used herein, the term “effective amount” or “effective dose” refers to the amount that can produce function or activity on humans and/or animals and can be accepted by human and/or animal.

As used herein, “pharmaceutically acceptable” component is a substance which can be applied to humans and/or mammals without undue adverse side effects (such as toxicity, irritation and allergic reactions), that is to say, substances of reasonable benefit/risk ratio. The term “pharmaceutically acceptable carrier” refers to a carrier for administration of a therapeutic agent, including various excipients and diluents.

The carriers of the pharmaceutical compositions of the present invention include (but are not limited to): saline, buffer solution, glucose, water, glycerol, ethanol, and combinations thereof. Pharmaceutical preparations usually should match the method of administration. The pharmaceutical compositions of the invention may be prepared in the form of injections, for example, prepared with saline or aqueous solutions containing glucose and other adjuvants by conventional methods. The pharmaceutical compositions preferably manufactured under sterile conditions. The amount of active ingredient administered is a therapeutically effective amount. Pharmaceutical formulations of the present invention may also be prepared into sustained release formulations.

The effective amount of mesenchymal vessel-layer cells and mesenchymal progenitor cells of the present invention may vary with the mode of administration and the severity of the diseases being treated. A preferred option of the effective amount may be based on a variety of factors determined by those skilled in the art (e.g., via clinical trials). The factors include, but are not limited to: the pharmacokinetic parameters such as bioavailability, metabolism, half-life and the like; the severity of the patient's disease to be treated, body weight or immune status of a patient, the route of administration, etc.

The pharmaceutical compositions of the present invention are preferably intraarticular injection reagents. In another preferred embodiment, the concentration of the mesenchymal progenitor cells of intraarticular injection reagents is 10⁵-10⁹/mL/ml, preferably 10⁶-10⁸/mL, more preferably 5×10⁶-5×10⁷/mL.

The present invention also provides a method of using the pharmaceutical compositions of the present invention, in a particular embodiment, comprising the following steps:

(1) administering mesenchymal vessel-layer cells to a subject in need; and

(2) administering mesenchymal progenitor cells to a subject in need, the preferred administration time is one month, and/or three months after step (1).

In the present invention, mesenchymal progenitor cells are administered to a subject in need, the preferred administration site is joint cavity of the said subject, thereby stimulating the differentiation of progenitor cells to repair the lesion.

The Main Merits of the Present Invention Comprise:

1. The present invention prepare injection from fat-derived mesenchymal progenitor cells, sodium hyaluronate and human serum albumin, as fat-derived mesenchymal progenitor cell solvent, which can be filled to the articular cartilage injury site by intra-articular injection to treat large area cartilage defects.

2. The present invention uses optimized formulation volume and component concentration, which has shown an unexpected therapeutic effect and the fat-derived mesenchymal progenitor cells in the formulation can survive for an unexpectedly long time.

The present invention will be further illustrated below with reference to the specific examples. It should be understood that these examples are only to illustrate the invention but not to limit the scope of the invention. The experimental methods with no specific conditions described in the following examples are generally performed under the conventional conditions, such as conditions illustrated in Sambrook et al, Molecular Cloning: A Laboratory Manual (New York: Cold Spring Harbor Laboratory Press, 1989), or according to the manufacture's instructions.

### Example 1 Cell Separation, Purification and Culture

a) washing fat tissue (to remove blood cells): same amount of saline was added to the fat-containing centrifuge tube, the lid was tighten and shook for 3 min to fully wash the fat tissue, then stood for 3-5 min to separate different phase, the lower aqueous phase was absorbed; the above operation was repeated for three times until the lower liquid was comparatively clear.

b) collagenase digestion: after the saline was removed, equal volume preheated DMEM containing 0.1% collagenase I was added, and placed in a constant temperature oscillator in 37° C. and digested for two hours under 200 rpm. The centrifuge tube was shaken for 5˜10 seconds (making the fat and collagenase I fully touch) every 15 min.

c) precipitation collection: after digestion, centrifuged for 10 min at 2000 rpm, the upper digested fat was removed, the depositions in underlying layer of two tube was collected into a new centrifuge tube, and DMEM was added to 50 ml, then centrifuged at 1000 rpm for 8 min to wash for once.

d) filtration and counting: DMEM was added to 50 ml, mixed, filtered to remove undigested tissue blocks by 100 μm filter, DMEM was added to 50 ml, and absorbed for 1 ml to count cell amount and vitality.

e) inoculating cultivate: washed once by centrifugation at 1000 rpm for 8 min, the inoculation density was adjusted according to the amount of cells counted to inoculated into T75 culture bottle (inoculation density: generally every T75 flask was inoculated with 12 ml of fat from liposuction, i.e. cells isolated from 50 ml liposuction fat were inoculated into four T75 culture bottle), then cultivated at 37° C. and 5% CO₂.

f) passage: the cells adhered after 1-2 days inoculation. After cultivated for 3 days, a few adherent mesenchymal stem cells started to appear, and adherent cells formed colonies after cultivated for 5-7 days. Trypsin EDTA solution was used for digestion and passaging, and 2 ml was added to each T75 culture bottle, of which the digestion time was 1.5-2.5 min, and the cells were collected for cell counting, and passaged at 5×10³/cm² (i.e., passaged at a ratio of 1:1-2 according to the primary adherent conditions), the cells grow faster after passage and can be passaged again in three days. The cells were passaged at a ratio of 1:2-3 according to the cell growth, and P3-P7 passage cells were collected for treatment or preparation of pharmaceutical formulations.

### Example 2 the Identification of Fat Progenitor Cells

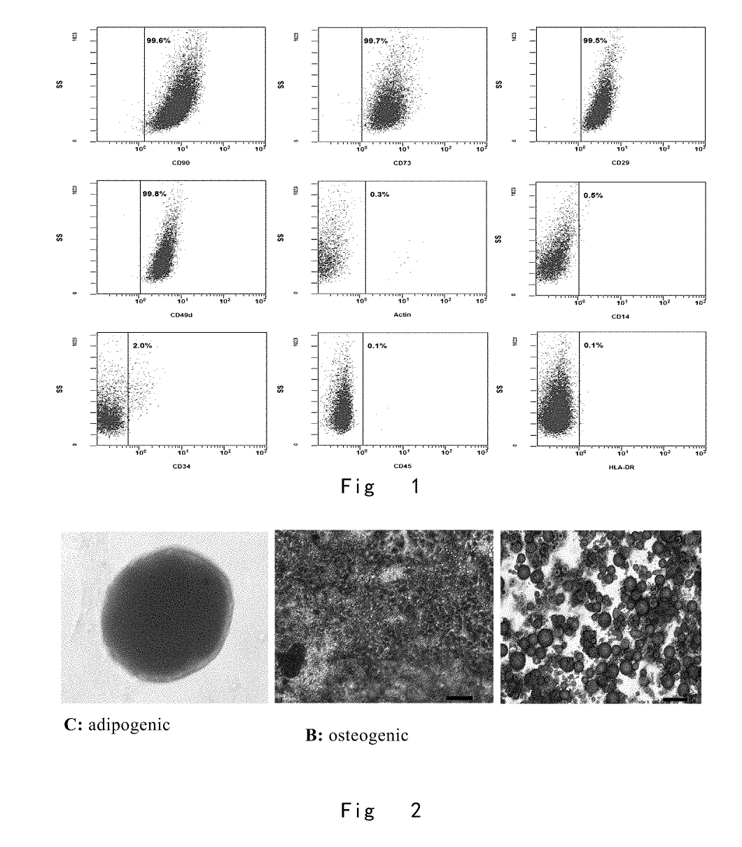

The fat progenitor cells cultured in the example 1 were centrifuged and resuspended. After cell count, the cell concentration was adjusted to 1×10⁸/L, and cells were reacted with human anti-CD34, CD45, CD29, CD73, CD90, CD105, Actin, CD14, and HLA-DR monoclonal antibody respectively at room temperature for 30 min, then resuspended with PBS. Flow cytometry was used for the detection (FIG. 1)

| | Surface antigen | | | | | | | | |
|---|---|---|---|---|---|---|---|---|---|
| | CD34 | CD45 | CD29 | CD73 | CD90 | CD49d | Actin | CD14 | HLA-DR |
| results | 2.0% | 0.1% | 99.5% | 99.7% | 99.6% | 99.8% | 0.3% | 0.5% | 0.1% |

Conclusion: the cell surface antigen marker expression analysis of the fat-derived progenitor cells by flow cytometry showed that the cells were of high purity.

### Example 3 Test of Cartilage, Osteogenesis, Adipogenic Differentiation Ability of Fat-Derived Mesenchymal Progenitor Cells

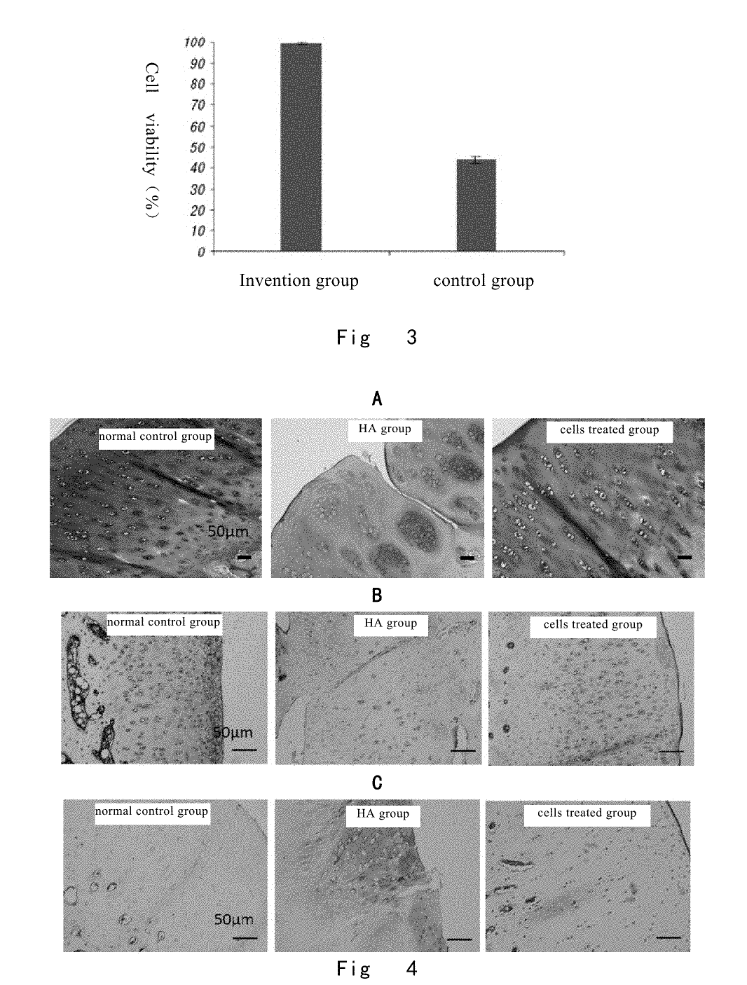

The cells cultured in Example 1 are used as the present invention group, and were subjected to cartilage, osteogenesis, and adipogenic differentiation ability test. Arctic blue staining after differentiation of the cartilage in vitro for 3 to 4 weeks showed that the cells cultured in Example 1 had the ability to differentiate into cartilage in vitro (FIG. 2A); The alizarin red staining after differentiation of the bone in vitro for 3 to 4 weeks showed that the cells cultured in example 1 had an ability to differentiate into the bone in vitro (FIG. 2B); The Oil red O staining after differentiation of the fat in vitro for 3 to 4 weeks showed that the cells cultured in Example 1 had an ability to differentiate into the fat in vitro (FIG. 2B)

### Example 4 Detection of Cytokines Expressed by Fat-Derived Mesenchymal Progenitor Cells

The fat-derived mesenchymal progenitor cells cultured in Example 1 were resuspended after centrifugation, and were inoculated by adjusting cell density. After 48 h, the supernatant was collected and the cytokines TGF-β1, HGF and VEGF were detected. The results were shown in the following table.

| cytokines | TGF-β1 | HGF | VEGF |
|---|---|---|---|
| results(pg/ml/106 cells) | 1256 | 9663 | 747 |

Conclusion: The fat-derived mesenchymal progenitor cells can express TGF-β1, HGF and VEGF,

### Example 5 Preparation of Knee Articular Cartilage Repair Injections

Cells were digested with Trypsin EDTA to prepare cell suspensions, and washed three times with saline to remove residual liquid. According to the area of articular cartilage injury, the appropriate cells amount of 1×10⁶ cells/cm² articular cartilage injury area was prepared, and the cells were resuspended by using sodium hyaluronate injection and mixed with human serum albumin solution to prepare the final product composition. Wherein the cells used were cells prepared by the example 1.

It was found after study that after resuspended with 20% sodium hyaluronate injection, the cells were mixed with 1% human serum albumin at a 3:1 volume ratio, thus ensuring 8 hours of effective storage time of fat-derived mesenchymal progenitor cells, and leading the form of an effective biological scaffold structure, so that the final product would be easier to fill the cartilage defect site and maintain a certain degree of viscosity to better combine into cartilage defect site.

The effect of the final product dosage form of the fat-derived mesenchymal progenitor cells on cell viability was shown in FIG. 3. In the figure, the cell viability of the final product dosage form of the fat-derived mesenchymal progenitor cells by the Trypan blue method was shown in the left side, while the right side has shown the cell viability after fat-derived tissue mesenchymal progenitor cells was directly combined with sodium hyaluronate injection for 8 hours. It can be seen that the dosage form of the present invention can well maintain the survival rate of fat-derived mesenchymal progenitor cells.

### Example 6 the Treatment of Human Fat-Derived Mesenchymal Progenitor Cells Composition on New Zealand White Rabbit Articular Cartilage Injury

30 rabbits were divided into three groups, while 10 in each group. The first group had not been treated, and the second and third group was preformed right lower limb knee anterior cruciate ligament excision and medial meniscus was removed to form cartilage injury model after 6 weeks. After operation, the second and the third groups was injected with sodium hyaluronate and fat-derived mesenchymal progenitor cell composition at the sixth week, ninth weeks and twelfth weeks respectively. The animals were sacrificed at the 16th week after operation, and safranin O/fast green staining, type II collagen and MMP-13 immunohistochemistry were performed.

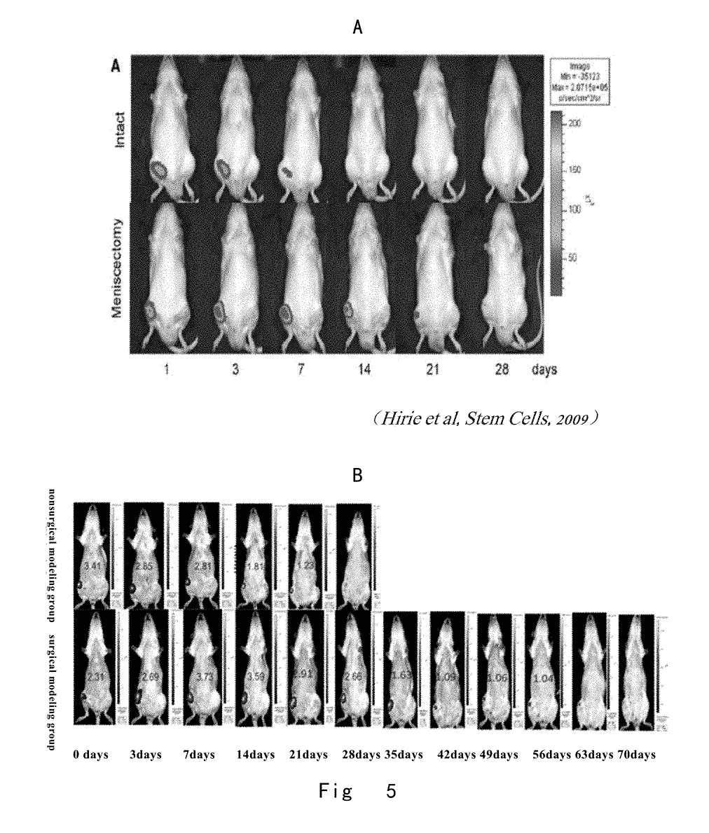

Conclusion: The human fat-derived mesenchymal progenitor cell composition of the present invention can regenerate damaged cartilage, and the safranin O/fast green staining has shown the significant regeneration of cartilage matrix (FIG. 4A); cartilage type II collagen hyperplasia has significantly grown (FIG. 4B) and cartilage degrading enzyme MMP-13 secretion was inhibited (FIG. 4C).

### Example 7 Tracer Experiment of Human Fat-Derived Mesenchymal Progenitor Cells Composition after SD Rat Knee Joint Injection

The cell concentration was adjusted to 1×10⁶/mL in serum-free medium: DiD cell marker solution was mixed into the cell suspension at a ratio of 1:100, i.e., 10 μl of cell marker solution was added to the every 1 mL of cell suspension, and slightly pipette blown to mix; and the cells were incubated at 37° C. for 50 minutes. The labeled cell suspension was centrifuged at 37° C. and 1500 rpm for 15 minutes. The supernatant was removed and the cells were resuspended in serum-free medium at 37° C.; the steps 4 and 5 were repeated to wash the cells twice or more; for 10 minutes after wash, fluorescence detection was performed.

The right hind limb medial meniscuses of 2-month-old SPF grade SD rats were removed to form model. All the experimental rats were given a right hind limb knee joint cavity injection immediately after modeling. Twenty rats were randomly divided into two groups, in which the first group was the control group, and was injected with 100 ul sodium hyaluronate solution; the second group was the experimental group, and was injected with 100 ul (2.5×10⁶) human fat-derived mesenchymal progenitor cells composition stained with membrane dye DiD; and the third group was model group without operation, and was injected with 100 ul (2.5×10⁶) human fat-derived mesenchymal progenitor cells composition stained with membrane dye DiD. The small animal imager (PerkinElmer company) was used to detect residual cells in the rat knee every week.

Conclusion: The 2.5×10⁶ human fat-derived mesenchymal progenitor cells of the invention can survive for about 10 weeks in SD rats, and survive for 4 weeks in non-operative group (FIG. 5B), and the survival time was both better than that of 5×10⁶ human synovial membrane-derived mesenchymal progenitor cells in Rat knee joint injection experiment by Japanese group (about 4 weeks, FIG. 5A).

### Example 8 Repair of Knee Articular Cartilage in Clinic

18 patients with articular cartilage injury were taken 30-50 ml autologous fat after the approval of ethics committee and signed the informed consent. The autologous fat was used to prepare fat-derived mesenchymal progenitor cells composition, and 3 ml of composition was injected into the articular cavity with cartilage injury, self control observed the therapeutic effect of 3 months, 6 months, and 12 months after injection.

Patient position: supine position, body muscles relaxed

Joint: Straight or slightly curved

Injective point: injected in knee eye inside and outside edge of the patella

Patients were in supine position, and the knee was kept straight. The intersection of the upper edge of the patella and the inner and outer edge of the patella was two points, oblique to the center of patellofemoral joint, and punctured at 45°. Knee was kept flexed at about 30°, and injected vertically from the medial patellar tendon below the patella or lateral articular space. The final composition product was slowly injected into the cavity of the knee joint and kept for 30 minutes after injection, and then normal activity could be carried out.

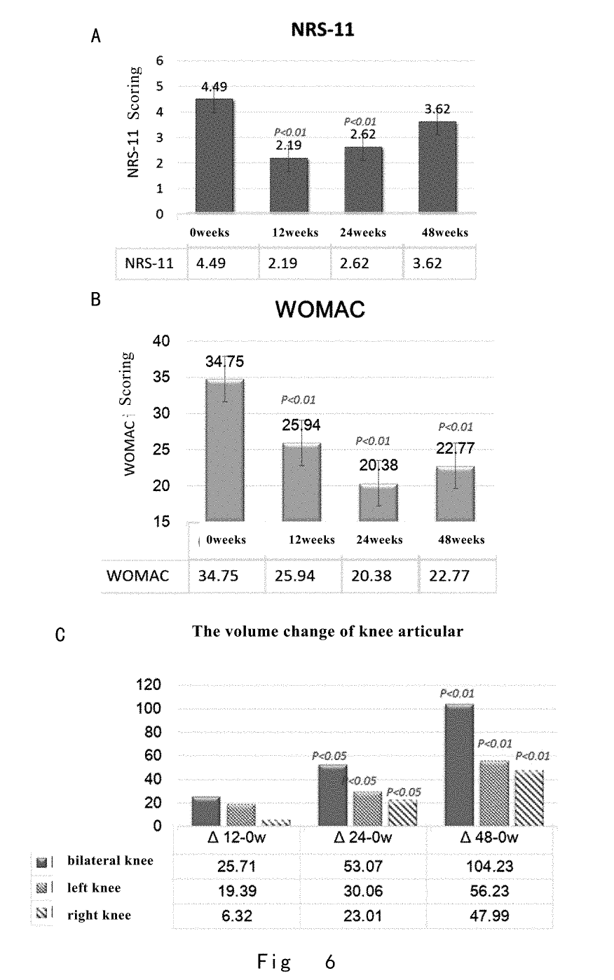

The patients were followed up at twelfth, twenty-fourth, and forty-eighth weeks after treatment. The results showed that fat-derived mesenchymal progenitor cells composition can significantly reduce patient's joint pain, and the pain score NRS-11 decreased significantly (FIG. 6A); the fat-derived mesenchymal progenitor cells composition improved joint activity function, and dysfunction of joint activity WOMAC scores decreased significantly (FIG. 6B); the volume of articular cartilage measured by MRI increased significantly (FIG. 6C).

Conclusion: the compositions of fat-derived mesenchymal progenitor cells and sodium hyaluronate injection have a significant effect on the treatment of articular cartilage defects.

### Example 9 Repair of Knee Articular Cartilage in Clinic

Research Methods:

Subjects were treated with intra-articular injection therapy after knee arthroscopy operation. The subjects of the cell therapy group were treated with the formulation of the example 5 (of which the volume of single injection was 3 ml) at the first and fourth weeks and treated with ARTZ therapy at the second and third weeks. The subjects of control group were treated four times with ARTZ (sodium hyaluronate injection, 2.5 ml: 25 mg) therapy once a week. The total observation time of the study was 12 months and the efficacy and safety were assessed at 8, 24, 36, and 48 weeks after the first treatment. The evaluation indexes of efficacy comprise WOMAC score, VAS score, MRI detection of cartilage volume (24 weeks, 36 weeks, and 48 weeks), arthroscopy observation (24 weeks), etc. The evaluation indexes of safety comprise adverse events and related laboratory tests during the whole study period.

Case 1 and case 2 patients were treated with the knee arthroscopic debridement and intra-articular injection of formulation of the example 5. Patients were treated with arthroscopy operation and knee intra-articular injection of formulation of the example 5 at the first week, ARTZ knee intra-articular injection in the second and third week, and knee intra-articular injection by the example 5 formulations in the fourth week.

The treatment result was as follows:

Case 1 patient

| WOMAC score | baseline | 8 weeks | 24 weeks | 36 weeks |
|---|---|---|---|---|
| The total score | 53 | 56 | 22 | 15 |
| Pain score | 10 | 9 | 4 | 2 |
| Rigidity score | 4 | 2 | 0 | 0 |
| Daily activities score | 39 | 45 | 18 | 13 |
| SF-36 sore | 87 | 80 | 89 | 87 |
| VAS pain score | 7 | 7.5 | 6 | 6.9 |

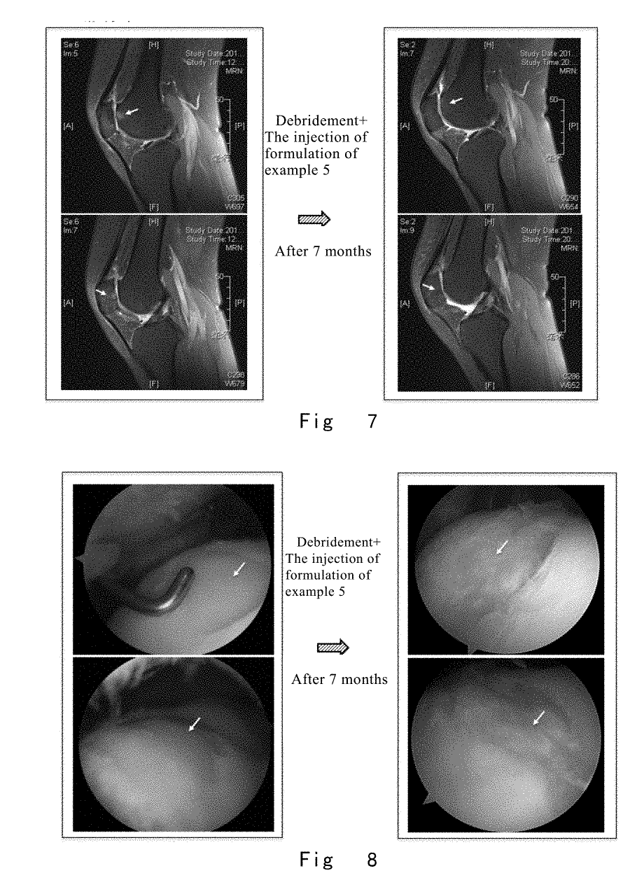

The results of MRI detection were showed in the following table and FIG. 7, the results showed that after treatment, the patient's knee osteoarthritis was significantly relieved, the patient's subchondral bone lesions was improved significantly, and the volume of cartilage increased from 26.948 cm² to 30.621 cm².

| Cartilage defect | baseline | 7 month |
|---|---|---|
| Volume (cm³) | 26.948 | 30.621 |

The results of arthroscopic observation were shown in FIG. 8. FIG. 8 showed that there was obvious cartilage like tissue hyperplasia in the patient's cartilage defect sites after treatment.

Case 2 patient

| WOMAC score | baseline | 8 weeks | 24 weeks | 36 weeks |
|---|---|---|---|---|
| The total score | 53 | 56 | 22 | 15 |
| Pain score | 10 | 9 | 4 | 2 |
| Rigidity score | 4 | 2 | 0 | 0 |
| Daily activities score | 39 | 45 | 18 | 13 |
| SF-36 sore | 87 | 80 | 89 | 87 |
| VAS pain score | 7 | 7.5 | 6 | 6.9 |

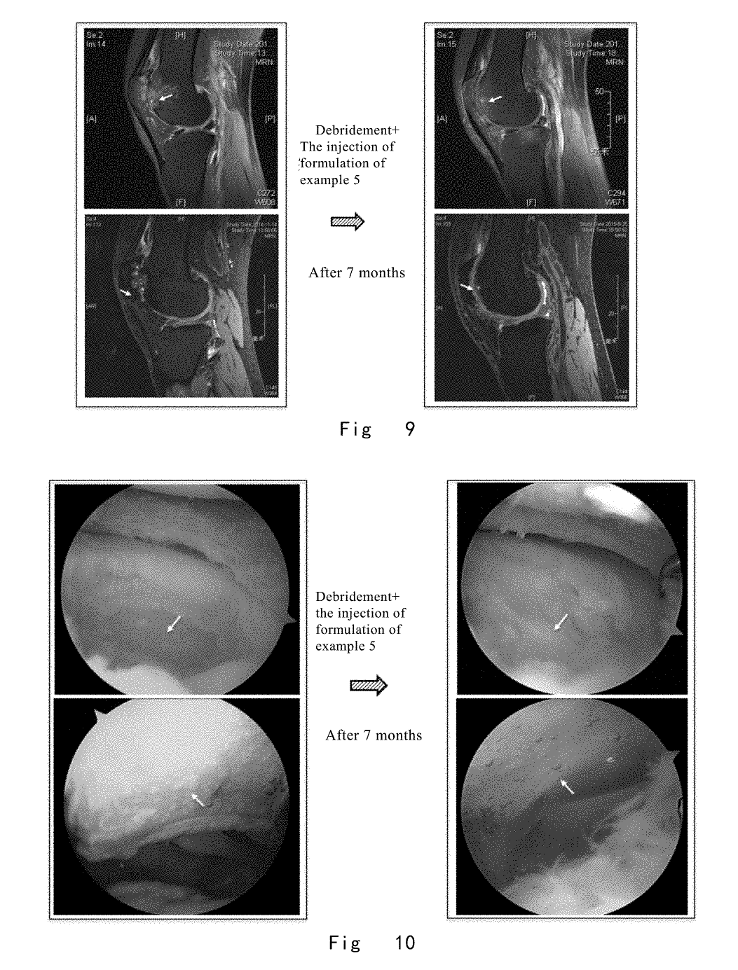

The results of MRI detection were showed in the following table and FIG. 9, the results showed the patient's knee osteoarthritis was significantly relived, the patient's subchondral bone lesions improved significantly, and the volume of cartilage increased from 18.365 cm² to 20.831 cm².

| The volume of cartilage | baseline | 7 month |
|---|---|---|
| Volume(cm³) | 18.365 | 20.831 |

The results of arthroscopic observation were shown in FIG. 10. FIG. 10 showed that there was obvious cartilage like tissue hyperplasia in the patient's cartilage defect sites after treatment.

In case 3, the patient was treated with knee arthroscopic debridement and ARTZ knee intra-articular injection. Patients were treated with arthroscopic debridement and ARTZ knee intra-articular injection at the first week, and ARTZ knee intra-articular injected at the 2nd, 3rd and 4th week.

| WOMAC score | baseline | 8 weeks | 24 weeks |
|---|---|---|---|
| The total score | 38 | 47 | 34 |
| Pain score | 6 | 8 | 7 |
| Rigidity score | 1 | 2 | 2 |
| Daily activities score | 31 | 37 | 25 |
| SF-36 sore | 78 | 109 | 96 |
| VAS pain score | 4.5 | 6.9 | 6 |

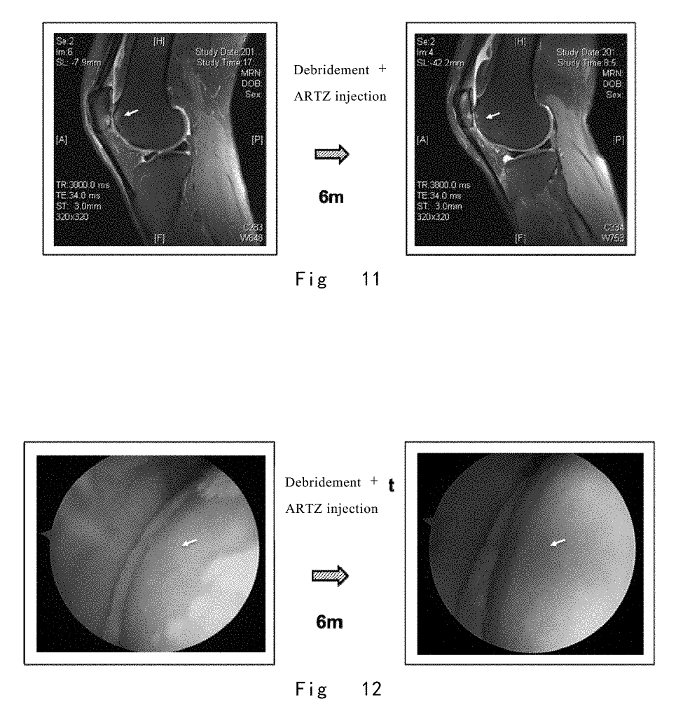

The results of MRI detection were showed in the following table and FIG. 11. The results showed the patient's knee osteoarthritis has not shown significant alleviation, the subchondral bone lesions has shown no significant improvement, and the volume of cartilage has not shown significant increase.

| Cartilage defect | baseline | 24 month |
|---|---|---|
| volume(cm³) | 19.111 | 19.626 |

The results of arthroscopic observation were shown in FIG. 12. There was no obvious cartilage like tissue hyperplasia in the patient's cartilage defect sites after treatment.

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A composition for treating articular cartilage defects, wherein the composition comprises a therapeutically effective amount of fat-derived mesenchymal progenitor cells, human serum albumin solution, and sodium hyaluronate.

2. The composition of claim 1, wherein the mesenchymal progenitor cells have one or more features selected from the group consisting of:
(i) more than 95% of cells having surface antigen CD90;
(ii) more than 95% of cells having surface antigen CD73;
(iii) more than 95% of cells having surface antigen CD29;
(iv) more than 95% of cells having surface antigen CD49d.

3. The composition of claim 1, wherein said mesenchymal progenitor cells have one or more features selected from the group consisting of:
(v) less than 2% of cells having surface antigen CD34;
(vi)) less than 1% cells having surface antigen CD45;
(vii) less than 0.3% f of cells having surface antigen Actin;
(viii) less than 0.5% of cells having surface antigen CD14;
(ix) less than 0.1% of cells having surface antigen HLA-DR.

4. The composition of claim 1, wherein the concentration of fat-derived mesenchymal progenitor cells in the composition is 10⁵-10⁹/mL.

5. The composition of claim 1, wherein the fat-derived mesenchymal progenitor cells further express cytokines, and the cytokines are selected from the group consisting of TGF-β1, HGF, VEGF, or the combinations thereof.

6. The composition of claim 1, wherein the volume ratio of sodium hyaluronate to human serum albumin in said composition is 1-5:0.8-1.2, preferably 2-4:1, more preferably 2.5-3.5:1.

7. The composition of claim 1, wherein the composition is a unit dosage form, and the volume of the unit dosage form is ≤4 mL, preferably ≤3 mL.

8. The composition of claim 1, wherein the human serum albumin solution is 0.5-2 (v/v) % human serum albumin; preferably 0.8-1.5 (v/v %).

9. The composition of claim 1, wherein the sodium hyaluronate is solution, preferably 15-25 wt % sodium hyaluronate aqueous solution.

10. The composition of claim 1, wherein more than 98% of the cells have surface antigen CD90; and/or
more than 98% of the cells have surface antigen CD73; and/or
more than 98% of the cells have surface antigen CD29; and/or
more than 98% of the cells have surface antigen CD49d.

11. The composition of claim 1, wherein less than 1% of the cells have surface antigen CD34, and/or
less than 0.5% of the cells have surface antigen CD45.

12. The composition of claim 1, wherein, the amount of cytokine TGF-β1 expressed by the fat-derived mesenchymal progenitor cells is 1000-1300 pg/ml/10⁶ cell; and/or
the amount of cytokine HGF expressed by the fat-derived mesenchymal progenitor cells is 9000-10000 pg/ml/10⁶ cells; and/or
the amount of cytokine VEGF expressed by the fat-derived mesenchymal progenitor cells is 300-800 pg/ml/10⁶ cells.

13. A method for preparing the composition of claim 1, wherein, said method comprises steps: mixing fat-derived mesenchymal progenitor cells, human serum albumin solution and sodium hyaluronate to prepare composition.

14. A formulation for treating articular cartilage defects, wherein the formulation comprises the composition of claim 1 as an active ingredient.

15. An agent combination or a kit, wherein, comprising:
a. fat-derived mesenchymal progenitor cells;
b. 15-25% sodium hyaluronate solution;
c. 0.5-2% human serum albumin solution;
and d. specification, in which the use scheme is described;
and the use scheme comprises the step: mixing components a, b, and c for treating patients with articular cartilage defects.
